(19) United States
(12) Patent Application Publication
(10) Pub. No.: US 2011/0160172 A1
(43) Pub. Date: Jun. 30, 2011

(54) FLUOROQUINOLONE DERIVATIVES FOR OPHTHALMIC APPLICATIONS

(75) Inventors: Geoffrey Robert Owen, Southlake, TX (US); Amy C. Brooks, Burleson, TX (US); Lina Bernal-Perez, Fort Worth, TX (US); David W. Stroman, Irving, TX (US); Joseph J. Dajcs, Fort Worth, TX (US)

(73) Assignee: ALCON RESEARCH, LTD., Fort Worth, TX (US)

(21) Appl. No.: 13/043,118

(22) Filed: Mar. 8, 2011

Related U.S. Application Data
(63) Continuation of application No. 12/371,880, filed on Feb. 16, 2009.
(60) Provisional application No. 61/029,180, filed on Feb. 15, 2008.

(51) Int. Cl. A61K 31/4709; A61K 31/56; A61K 31/573; A61P 29/00; A61P 31/00; A61P 27/02

(52) U.S. Cl. 514/171; 514/300

(57) ABSTRACT

The present invention relates to fluoroquinolone derivatives having enhanced ocular penetration characteristics and/or antimicrobial activity, and to compositions comprising such derivatives. The derivatives and compositions are particularly well suited for treating ophthalmic bacterial infections. The present invention more particularly relates to the discovery that a 2-methyl substitution on a diazabicyclo group attached to a fluoroquinolone ring system produces improved permeability characteristics, and that a 5-amino substitution on a fluoroquinolone ring system results in improved anti-microbial activity.

### CROSS-REFERENCE TO RELATED APPLICATION

This application is a Continuation (CON) of co-pending U.S. patent application Ser. No. 12/371,880, filed Feb. 16, 2009, priority of which is claimed under 35 U.S.C. §120, the contents of which are incorporated herein by reference. This application also claims priority under 35 U.S.C. §119 to U.S. Provisional Patent Application No. 61/029,180 filed Feb. 15, 2008, the entire contents of which are incorporated herein by reference.

### TECHNICAL FIELD OF THE INVENTION

The present invention relates generally to fluoroquinolone derivatives for ophthalmic applications. The present invention particularly relates to fluoroquinolone derivatives having improved ocular penetration properties and/or antimicrobial activity.

### BACKGROUND OF THE INVENTION

The use of fluoroquinolone compounds to treat infections, including ophthalmic infections, is considered a state of the art treatment. Alcon Laboratories, Inc. markets a topical ophthalmic composition called VIGAMOX® ophthalmic solution that contains the fluoroquinolone antibiotic moxifloxacin (0.5%). Other commercially available fluoroquinolone antibiotics include gatifloxacin, levofloxacin, ofloxacin, ciprofloxacin, norfloxacin, and lomefloxacin. However, despite the general efficacy of the fluoroquinolone therapies currently available, there remains a need for improved antibiotic-based compositions and methods of treatment that are more effective than existing antibiotics against key ophthalmic pathogens.

It is generally desirable to use the minimum quantity of an antimicrobial compound necessary to achieve desired effects. This is because undesirable side-effects such as toxicity or irritation are more probable when higher concentrations of an antimicrobial are used at a delivery site through the use of, for example, high concentration compositions, more frequent dosing, or longer-duration treatment. Unfortunately, while the use of lower concentrations of antimicrobial compounds generally helps to reduce the potential for undesirable effects, this practice increases the risk that the compounds may not achieve the required level of antimicrobial effect. Also, microbial resistance can develop quickly if antimicrobial compounds are not used at a sufficient concentration. Therefore, the use of compounds having good antimicrobial activity is desirable as these compounds may be used at lower concentrations relative to compounds with lower activity, reducing the incidence and risk of undesired side effects and while preventing the development of microbial resistance.

It is also desirable that antimicrobial compounds have good permeability characteristics (e.g., they rapidly diffuse into tissue to which they are applied). Antimicrobial compounds that are unable to penetrate tissues are generally not useful as topical agents. Also, the rate of permeation of antimicrobial agents is important, as antimicrobial compounds should possess the ability to both quickly treat the surface and deeper portions of infected tissues.

U.S. Pat. No. 4,990,517 entitled “7-(1-pyrrolidinyl)-3-quinolone- and -naphthyridonecarboxylic acid derivatives as antibacterial agents and feed additives” discloses certain fluoroquinolone compounds that are useful as antimicrobial agents. U.S. Pat. No. 6,716,830 entitled “Ophthalmic antibiotic compositions containing moxifloxacin” discloses fluoroquinolone compounds that are useful as ophthalmic antibiotics. Neither patent discloses a relationship between the structure of such compounds and their activity and/or permeability.

### BRIEF SUMMARY OF THE INVENTION

The invention relates to fluoroquinolone derivatives, and the use of such derivatives to treat ophthalmic conditions. The present inventors have unexpectedly discovered that the fluoroquinolone derivatives of the present invention have improved antimicrobial activity and/or permeability in ocular tissues compared to known fluoroquinolone compounds used for ophthalmic applications. One fluoroquinolone derivative of the present invention has a measured permeability in ocular tissue that is approximately three times greater than the well-known ophthalmic anti-infective moxifloxacin. The increased antimicrobial activity and/or permeability of the fluoroquinolone derivatives disclosed herein make the compounds well suited for use as ophthalmic anti-infective agents. Relative to many other ophthalmic anti-infective agents, the compounds of the present invention may be used at lower concentrations and at a reduced dosing frequency. The compounds of the present invention having good permeability characteristics are also rapid acting and may be used as first-line anti-infective agents in acute infections.

The fluoroquinolone derivatives of the present invention may be used in various ophthalmic compositions disclosed herein. Such compositions are preferably sterile and have physiologically compatible properties, particularly with ocular tissue. Such ophthalmic compositions may be used in the treatment of ophthalmic infections including, but not limited to, conjunctivitis, keratitis, endophthalmitis, and blepharitis. Corneal ulcers may also be treated by compositions of the present invention.

The compositions of the present invention may optionally comprise in addition to a fluoroquinolone derivative an anti-inflammatory agent. Tissue infections frequently present with associated edema and inflammation, and the antimicrobial and anti-inflammatory compositions are useful in treating such infections.

The compositions of the present invention may also be used in the prophylaxis of infection following tissue trauma (including trauma resulting from surgical procedures). The fluoroquinolone and anti-inflammatory agent compositions are particularly useful in such prophylaxis, as inflammation is especially present following surgery or physical trauma to tissue.

### DETAILED DESCRIPTION OF THE INVENTION

The fluoroquinolone derivatives of the present invention have the following general formula:

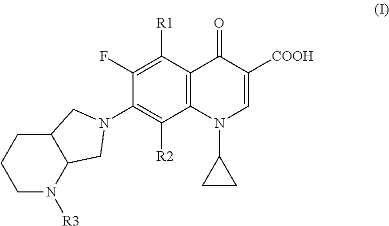

wherein:
R1 is H, amino, C1-C4 alkylamino, or C1-C4 dialkylamino

### R2 is F, OCH₃, or H;

R3 is CH₃, C2-C4 alkyl, or H; and
at least one of R1 and R3 is not H.

The present inventors have discovered that the substitution of a methyl or C2-C4 alkyl group at the position denoted by R3 in Formula (I) produces improved permeability characteristics relative to other fluoroquinolones such as moxifloxacin. In a preferred embodiment, this substitution is characterized as a 2-methyl substitution on the diazabicyclo group attached to the fluoroquinolone ring. Further, the inventors have discovered that an amino or substituted amino derivative at the R1 position in Formula (I) results in both improved antimicrobial activity and permeability characteristics relative to other fluoroquinolones. In a preferred embodiment, this substitution is characterized as a 5-amino substitution on the fluoroquinolone ring. Data presented in Examples 3 and 4 for several preferred compounds demonstrate the structure and activity/permeability correlation discovered by the inventors.

Preferred fluoroquinolone derivatives of the present invention are of the following two formulas. The first formula encompasses compounds that have enhanced permeability relative to known fluoroquinolone compounds and is as follows:

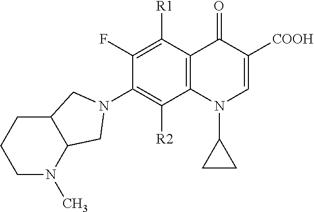

wherein:
R1 is H, amino, C1-C4 alkylamino, or C1-C4 dialkylamino; and
R2 is F, OCH₃, or H. The second formula encompasses compounds having enhanced anti-microbial activity relative to known fluoroquinolones:

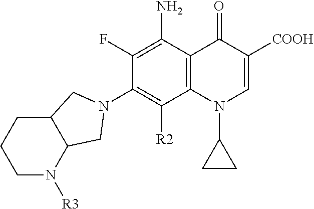

wherein:

### R2 is F, OCH₃, or H; and

R3 is CH₃, C2-C4 alkyl, or H.

The most preferred fluoroquinolone derivatives of the present invention are listed in TABLE 1 below (the substituent groups denoted in TABLE 1 refer to Formula (I)) and their structures are shown below TABLE 1.

TABLE 1

| Compound No. | R1 | R2 | R3 |
|---|---|---|---|
| 1 | H | F | CH₃ |
| 2 | H | H | CH₃ |
| 3 | NH₂ | F | H |
| 4 | NH₂ | OCH₃ | H |
| 5 | NH₂ | F | CH₃ |
| 6 | NH₂ | OCH₃ | CH₃ |
| 7 | H | OCH₃ | CH₃ |

Compound 1 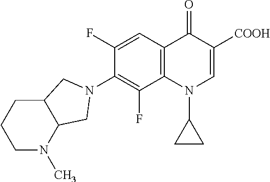
Compound 2 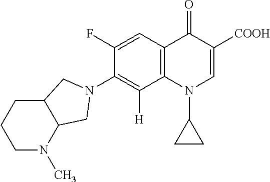
Compound 3 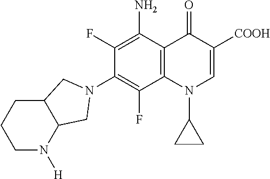
Compound 4 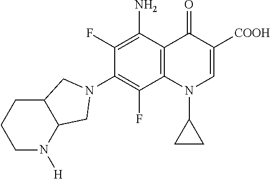
Compound 5 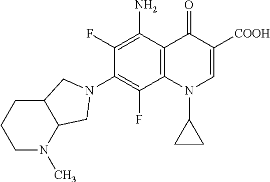
Compound 6 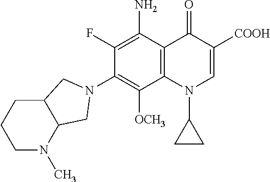
Compound 7

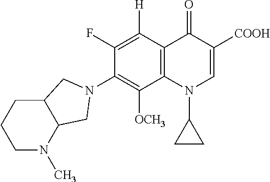

Relative to many other ophthalmic anti-infective agents, the compounds of the present invention may be used at lower concentrations and at a reduced dosing frequency. It is contemplated that the concentration of the active fluoroquinolone ingredient in the compositions of the present invention can vary, but is preferably 0.05 to 0.8 w/v % and more preferably 0.05-0.5 w/v %. The most preferred concentration range is from 0.05-0.3 w/v % and the most preferred concentration is about 0.3 w/v %. A pharmaceutically effective amount of a fluoroquinolone of the present invention is generally that concentration sufficient to produce a desired effect (such as achieving a MIC₉₀ level relative to the infectious organisms associated with the treated infection) at a reasonable benefit/risk ratio. The pharmaceutically effective amount may vary depending on such factors as the disease or infectious agent being treated, the particular formulation being administered, or the severity of the disease or infectious agent.

The derivatives described herein can be prepared using methods disclosed in U.S. Pat. No. 4,990,517 (Petersen et al.) which is herein incorporated by reference in its entirety, in combination with known synthetic methods available to those of skill in the art. The fluoroquinolone derivatives of the present invention comprise the pharmaceutically useful stereoisomers of the derivatives, as well as the pharmaceutically useful hydrates and salts of such derivatives and stereoisomers, and may be formulated with a pharmaceutically acceptable vehicle.

The invention is particularly directed toward treating mammalian and human subjects having or at risk of having a microbial tissue infection. Embodiments of the present invention are particularly useful for treating ophthalmic tissue infections. Infections of the eye can occur in all ocular tissues or fluids, and include diseases of the lid or lid margins (blepharitis), the conjunctiva (conjunctivitis), the cornea (microbial keratitis) and the deeper intraocular fluids or tissues (endophthalmitis). For each of these conditions, the choice of an appropriate topical antibiotic is important—the antibiotic must penetrate at an appropriate level into the affected tissues.

Embodiments of the present invention may also be used prophylactically to prevent infection of a tissue by an infectious agent. In such embodiments, a tissue at risk of infection is contacted with a composition of the present invention. Such prophylactic use is particularly useful during or following surgical procedures or physical trauma to tissue that create a risk of infection.

Compositions of the present invention may be utilized in various dosage regimens known to those of skill in the art. Such dosing frequency is maintained for a varying duration of time depending on the therapeutic regimen. The duration of a particular therapeutic regimen may vary from one-time dosing to a regimen that extends for a month or more. One of ordinary skill in the art would be familiar with determining a therapeutic regimen for a specific indication. Factors involved in this determination include the disease to be treated, particular characteristics of the subject, and the particular antimicrobial composition. Preferred dosage regimens of the present invention include, but are not limited to, once a day dosing, twice a day dosing, and three times a day dosing.

The compositions of the present invention may optionally comprise in addition to a fluoroquinolone derivative an anti-inflammatory agent. Such agents include, but are not limited to, steroids such as prednisolone, dexamethasone, hydrocortisone, and rimexolone, and non-steroidal compounds such as nepafenac, naproxen, ibuprofen, aspirin, PDE IV inhibitors (such as cilomilast), cytokine inhibitors, and other anti-inflammatory agents known to those of skill in the art.

In addition to a disclosed fluoroquinolone derivative, the compositions of the present invention optionally comprise one or more excipients. Excipients commonly used in pharmaceutical compositions include, but are not limited to, tonicity agents, preservatives, chelating agents, buffering agents, surfactants and antioxidants. Other excipients comprise solubilizing agents, stabilizing agents, comfort-enhancing agents, polymers, emollients, pH-adjusting agents and/or lubricants. Any of a variety of excipients may be used in compositions of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the fluoroquinolone derivative. In preferred embodiments, excipients are selected on the basis of their inertness towards the fluoroquinolone derivative.

Relative to ophthalmic formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, include ionic and nonionic surfactants, though nonionic surfactants are preferred, RLM 100, POE 20 cetylstearyl ethers such as Procol® CS20 and poloxamers such as Pluronic® F68. Suitable antioxidants include, but are not limited to, sulfites, ascorbates, butylated hydroxyanisole (BHA) and butylated hydroxytoluene (BHT).

The compositions set forth herein may comprise one or more preservatives. Examples of such preservatives include p-hydroxybenzoic acid ester, sodium chlorite, parabens such as methylparaben or propylparaben, alcohols such as chlorobutanol, benzyl alcohol or phenyl ethanol, guanidine derivatives such as polyhexamethylene biguanide, sodium perborate, or sorbic acid. In certain embodiments, the composition may be self-preserved that no preservation agent is required.

In preferred compositions a fluoroquinolone derivative of the present invention will be formulated for topical application to the eye in aqueous solution in the form of drops. The term “aqueous” typically denotes an aqueous composition wherein the composition is >50%, more preferably >75% and in particular >90% by weight water. These drops may be delivered from a single dose ampoule which may preferably be sterile and thus render bacteriostatic components of the composition unnecessary. Alternatively, the drops may be delivered from a multi-dose bottle which may preferably comprise a device which extracts any preservative from the composition as it is delivered, such devices being known in the art.

In other aspects, components of the invention may be delivered to the eye as a concentrated gel or a similar vehicle, or as dissolvable inserts that are placed beneath the eyelids. In yet other aspects, components of the invention may be delivered to the eye as ointments, water-in-oil and oil-in-water emulsions, solutions, or suspensions.

The compositions of the present invention are preferably isotonic or slightly hypotonic in order to combat any hypertonicity of tears caused by evaporation and/or disease. This may require a tonicity agent to bring the osmolality of the composition to a level at or near 210-320 milliosmoles per kilogram (mOsm/kg). The pH of the solution may be in an ophthalmic acceptable range of 3.0 to 8.0. The compositions of the present invention generally have an osmolality in the range of 220-320 mOsm/kg, and preferably have an osmolality in the range of 235-300 mOsm/kg. The ophthalmic compositions will generally be formulated as sterile aqueous solutions.

In certain embodiments, a fluoroquinolone derivative is formulated in a composition that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; guars, such as HP-guar and other guar derivatives, and carbomers, such as carbomer 934P, carbomer 941, carbomer 940 and carbomer 974P. Certain compositions of the present invention may be used with contact lenses or other ophthalmic products.

In certain embodiments, the compositions set forth herein have a viscosity of 0.5-100 cps, preferably 0.5-50 cps, and most preferably 1-20 cps. These viscosities insure that the product is comfortable, does not cause blurring, and is easily processed during manufacturing, transfer and filling operations.

The fluoroquinolone derivatives described herein may be included in various types of compositions having activities in addition to antimicrobial activity. Examples of such compositions include: ophthalmic pharmaceutical compositions, such as ocular lubricating products, artificial tears, astringents, topical disinfectants (alone or in combination with other antimicrobial agents such as, for example, betadine, etc.) and so on.

Preferred compositions are prepared using a buffering system that maintains the composition at a pH of about 3 to a pH of about 8.0, preferably 5.5-7.5, and most preferably 6.0-7.4. Topical compositions (particularly topical ophthalmic compositions) are preferred which have a physiological pH matching the tissue to which the composition will be applied or dispensed.

In the methods set forth herein, administration to a subject of a pharmaceutically effective amount of a composition of the present invention may be by various methods known to those of skill in the art, including, but not limited to, topical, subconjunctival, periocular, retrobulbar, subtenon, intraocular, subretinal, posterior juxtascleral, or suprachoroidal administration. In preferred embodiments, administration of a composition of the present invention is by topical administration to the ocular surface.

The following examples are presented to further illustrate selected embodiments of the present invention.

### Example 1

| Ingredient | |
|---|---|
| Fluoroquinolone derivative | 0.3% |
| Mineral Oil, USP | 2.0% |
| White petrolatum, USP | q.s. 100% |

### Example 2

| Ingredient | |
|---|---|
| Fluoroquinolone derivative | 0.3% |
| Boric acid | 0.3% |
| Sodium Chloride | 0.7% |
| Water | q.s. 100% |

### Example 3

### Permeability Studies

The permeability of compounds of the present invention and other fluoroquinolones such as moxifloxacin were determined in corneal tissue. The procedure used is summarized below.

Female New Zealand Albino rabbits were sacrificed by first anaesthetizing with ketamine (30 mg/Kg) and xylazine (6 mg/Kg) followed by an injection of an overdose of SLEEPAWAY* (sodium pentobarbital, 1 ml of a 26% solution) into the marginal ear vein. The intact eyes, along with the lids and conjunctival sacs were then enucleated and immediately stored in about 70 ml of fresh BSS PLUS® irrigation solution saturated with O₂/CO₂ (95:5).

Within one hour, the enucleated rabbit eyes were mounted in the modified perfusion chambers as described by Schoenwald R. N. and Huang H-S., “Corneal Penetration Behavior of β-Blocking Agents I: Physiochemical Factors,” Journal of Pharmaceutical Sciences, 72 (11) (November 1983). To accomplish this, the exposed cornea of the enucleated eye was carefully placed on a corneal holder, which maintained the cornea curvature and held the eye in place. Various tissues of the eye were dissected leaving the cornea, a small ring of scleral tissue, and the palpebral conjunctiva. The conjunctival and scleral tissue served as a gasket and permitted the cornea to be suspended within the corneal ring in the center of the perfusion chamber. The chamber was jacketed to maintain the cornea and the perfusion solution at 35° C. The corneal holder and chamber were made from acrylic plastic at the University of Iowa, Iowa City, Iowa.

The mounted cornea was clamped between the two cylindrical compartments of the perfusion chamber and 7.5 mls of BSS PLUS® irrigating solution was placed in the receiving (endothelial) side of the chamber with stirring and bubbling of the O₂/CO₂ (95:5) mixture. Then, 7 mls of the fluoroquinolone dissolved in BSS PLUS® irrigating solution at a concentration of 100 μMole (this is approximately 4 mg/100 mL) was added to the donor (epithelial) side of the chamber also with stirring and bubbling of the O₂/CO₂ (95:5) mixture. The difference in volume ensured that the cornea would not buckle during the course of the experiment.

Samples (150 μL) were withdrawn from the receiving chamber every 30 minutes over a five hour period, and an equal volume of irrigating solution was immediately added to the receiving chamber to maintain a constant volume. The concentration of fluoroquinolones in the samples was determined using reverse-phase HPLC. A Waters 2690 Separations Module fitted with a Waters Symmetry® C18 5 μm column and Waters 2487 Absorbance Detector were used with 40 μL injections and a 1.5 mL/min flow rate. Mixtures of acetonitrile and 31 mM phosphoric acid/sodium phosphate buffer at pH=3 were the mobile phase. The mobile phase (acetonitrile:phosphate buffer v/v) and UV detection wavelengths for each of the fluoroquinolones was determined beforehand. For example, for moxifloxacin 21:79, 295 nm; levofloxacin 12.6:87.4, 287 nm; and ofloxacin 12.6:87.4, 295 nm. After each permeability experiment, the cornea was trimmed of excess scleral tissue and conjunctiva, weighed and dried overnight over phosphorus pentoxide in a vacuum desiccator. It was then reweighed in order to determine the hydration level. A normal cornea has a hydration level of 76-80%. If the cornea is damaged in any way the hydration level rises. Data from corneas with hydration levels over 83% are discarded.

The rate of drug accumulation in the receiving (endothelial) chamber, and the apparent Permeability Coefficients (Papp) of the fluoroquinolones were then calculated as follows:

Permeability Coefficients:

$$Papp = \frac{Rate}{60 \times A \times {C}_{0}} ( cm \text{/} sec )$$

$$\begin{matrix}where \text{:} & Rate = µg \text{/} min ( slope ) \\ & A = Area ( {cm}^{2} ) = 1.087 {cm}^{2} \\ & {C}_{0} = Initial Concentration ( µg \text{/} mL )\end{matrix}$$

TABLE 2

| | MEAN | |
|---|---|---|
| | PERMEABILITY | |
| | COEFFICIENT | MEAN LAG TIME |
| COMPOUND | (× 10⁻⁷ cm/sec) | (min) |
| Moxifloxacin | 117 | 39 |
| Ofloxacin | 39 | 47 |
| Levofloxacin | 35 | 52 |
| Compound 1 | 311 | 12 |
| Compound 2 | 205 | 27 |
| Compound 3 (racemic) | 166 | 38 |
| Compound 3 (S, S) | 136 | 45 |

Compound 1 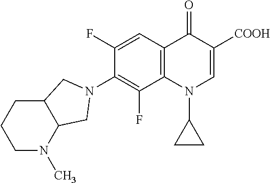
Compound 2 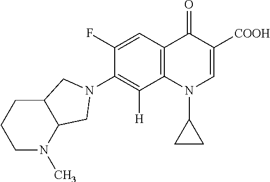
Compound 3

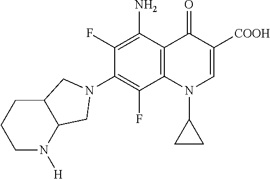

Compounds 1-3 (including both stereoisomeric forms of Compound 3) exceeded moxifloxacin's permeability and (with the exception of the S,S stereoisomer of Compound 3) had lower lag times relative to moxifloxacin. Compounds 1 and 2 having the 2-methyl substitution on the diazabicyclo ring had the best permeability and lag characteristics of the compounds studied.

### Example 4

### Antimicrobial Activity Studies

The activity of the compounds of the present invention was evaluated using an in vitro assay. The results are summarized in TABLE 3 below. Microorganisms were prepared from growth on agar media, in broth culture, or from thawed cryopreserved cultures. Fungal cultures were filtered to remove mycelial elements.

Using Mueller Hinton II Broth, 0.9% saline or other appropriate media, a suspension of each culture was prepared and adjusted to a turbidity equivalent to that of a 0.5 McFarland Standard. (Cultures adjusted to the 0.5 McFarland Standard generally contain approximately 1.0×10⁸ CFU/mL). The adjusted suspension for each culture was diluted 1:10 in the appropriate media so that the inoculum concentration approximates 1.0×10⁷ CFU/mL.

The antimicrobial agent to be tested was weighed and diluted with a volume of sterile distilled water calculated to yield the necessary starting concentration. Serial 1:2 dilutions of the antimicrobial agent in sterile distilled water were prepared (approximately 10-12 test concentrations comprise a reasonable experimental range). A known amount of each antimicrobial dilution was combined with an aliquot of agar medium calculated to yield the desired final antimicrobial test concentration and dispensed onto plates.

The amount of antimicrobial agent to be weighed was determined using the following equation:

$$Weight ( mg ) = \frac{Desired Volume ( mL ) \times Desired Concentration ( µg \text{/} mg )}{assay potency ( µg \text{/} mL )}$$

Antimicrobial stock solutions were prepared at concentrations 5-10 times the highest concentration to be tested (routinely 1024 μg/mL). Some antimicrobial agents of limited solubility may require lower concentrations or the addition of a few drops of 0.1 N NaOH (for anionic compounds) or 0.1 NH₂SO₄ (for cationic compounds) to aid in dissolution. The stock solution was diluted to appropriate concentration using sterile water to form a working solution. The working solution was diluted two-fold, in sterile water, until the desired concentration is obtained.

Mueller Hinton II agar was prepared as described by the manufacturer. Mueller Hinton II agar may be supplemented with 5% defibrinated sheep blood for testing streptococci. Agar was added to the serially diluted antimicrobial solution in each container. The solutions were mixed thoroughly and poured into petri dishes (about 25 mL/plate). The pH of the agar at 25° C. should be between 7.2 and 7.4.

At least one inocula control plate was inoculated (agar medium without antimicrobial agent) from each seed plate before inoculating test plates. Test plates were inoculated starting at the lowest concentration and proceeding to the highest concentration. After all test plates were inoculated, an additional control plate (agar medium without antimicrobial agent) was inoculated. The plates were incubated in a non-CO₂ incubator (or CO₂ incubator depending on growth requirements); 18-24 hours at 32-35° C. was sufficient for most organisms.

### MIC Results and Data Evaluation

All inoculation points on the agar surface were examined for the presence of growth as compared to the inoculum control plates. The results were summarized and use to determine Minimal Inhibitory Concentrations (MICs): The MIC is the lowest concentration of the antimicrobial agent which prevents visible growth of the organism (negative score). The results are summarized in TABLE 3 below.

TABLE 3

| | | | Compound 3 | Compound 3 | | |
|---|---|---|---|---|---|---|
| | Test Organism | Compound 2 | (S,S) | (Racemic) | Compound 1 | Moxifloxacin |
| 1 | Staphylococcus aureus | 0.125 | ≦0.03 | ≦0.03 | 0.06 | 0.06 |
| 2 | Staphylococcus aureus | ≦0.25 | ≦0.25 | 0.5 | ≦0.25 | ≦0.25 |
| 3 | MRSA | >2 | 1 | >2 | >2 | 2 |
| 4 | MRSA | >4 | 2 | 2 | 4 | 4 |
| 5 | MRSA | >2 | >2 | >2 | >2 | >2 |
| 6 | MSSA | >4 | 4 | >4 | >4 | >4 |
| 7 | Staphylococcus epidermidis | 2 | 0.5 | 0.06 | 0.25 | 0.06 |
| 8 | Staphylococcus epidermidis | >2 | 0.5 | >2 | >2 | 1 |
| 9 | MRSE | >2 | >2 | >2 | >2 | >2 |
| 10 | MRSE | >2 | >2 | >2 | >2 | >2 |
| 11 | MRSE | >4 | >4 | >4 | >4 | >4 |
| 12 | Staphylococcus haemolyticus | 1 | 0.125 | 0.125 | 0.25 | 0.5 |
| 13 | Staphylococcus haemolyticus | >4 | 1 | 1 | 2 | 2 |
| 14 | Staphylococcus haemolyticus | >4 | >4 | >4 | >4 | >4 |
| 15 | Enterococcus faecalis | 1 | 0.125 | 0.125 | 0.5 | 0.5 |
| 16 | Enterococcus faecalis | ≦0.25 | 0.5 | 1 | >2 | ≦0.25 |
| 17 | Enterococcus faecalis | >4 | >4 | >4 | >4 | >4 |
| 18 | Enterococcus faecalis | >2 | >2 | >2 | >2 | >2 |
| 19 | Streptococcus pneumoniae | 0.5 | 0.125 | ≦0.03 | 0.125 | 0.25 |
| 20 | Streptococcus pneumoniae | ≦0.25 | ≦0.25 | 0.5 | 0.5 | ≦0.25 |
| 21 | Streptococcus mitis | ≦0.25 | ≦0.25 | 0.5 | 0.5 | ≦0.25 |
| 22 | Streptococcus mitis | 0.5 | 0.125 | 0.06 | 0.25 | 0.125 |
| 23 | Streptococcus mitis | >4 | 1 | 1 | 2 | 4 |
| 24 | Corynebacterium amycolatum | ≦0.03 | 0.5 | 0.06 | 0.06 | 0.03 |
| 25 | Corynebacterium amycolatum | >4 | >4 | >4 | >4 | >4 |
| 26 | Acinetobacter baumannii | ≦0.25 | ≦0.25 | 0.5 | 0.5 | ≦0.25 |
| 27 | Acinetobacter junii | ≦0.25 | ≦0.25 | ≦0.25 | ≦0.25 | ≦0.25 |
| 28 | Acinetobacter junii | 0.25 | 0.06 | 0.125 | ≦0.03 | 0.25 |
| 29 | Acinetobacter junii | >4 | >4 | >4 | 2 | >4 |
| 30 | Enterobacter hormaechei | 0.25 | ND | 0.5 | ND | ND |
| 31 | Enterobacter hormaechei | >4 | 4 | 4 | 4 | >4 |
| 32 | Escherichia coli | 0.25 | 0.06 | 0.06 | 0.06 | 0.125 |
| 33 | Escherichia coli | ≦0.25 | ≦0.25 | ≦0.25 | ≦0.25 | ≦0.25 |
| 34 | Escherichia coli | >4 | >4 | >4 | >4 | >4 |
| 35 | Morganella morganii | 1 | 0.125 | 0.5 | 0.5 | 0.5 |
| 36 | Morganella morganii | 2 | 1 | 1 | 2 | 4 |
| 37 | Pseudomonas aeruginosa | >4 | >4 | >4 | >4 | >4 |
| 38 | Pseudomonas aeruginosa | >4 | 0.5 | 2 | 2 | 4 |
| 39 | Serratia marcescens | 4 | 0.5 | 0.5 | 1 | 2 |
| 40 | Serratia marcescens | 4 | 1 | 1 | 1 | 2 |
| 41 | Stenotrophomonas maltophilia | >4 | 2 | 4 | >4 | >4 |
| 42 | Stenotrophomonas maltophilia | 2 | 0.125 | ≦0.03 | 0.25 | 0.125 |

Compared to moxifloxacin, compound 3 (both the racemic and isomeric preparations), which has a 5-amino substitution on the fluoroquinolone ring system, showed increased antimicrobial activity against most microorganisms. Compound 1 showed somewhat better antimicrobial activity against certain microorganisms compared to moxifloxacin, and performed comparably when taken as a whole. Compound 2 generally did not perform as well as moxifloxacin or Compounds 1 and 3 in the antimicrobial activity test.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A topical ophthalmic pharmaceutical composition comprising:
a pharmaceutically effective amount of one or more compounds of the following formula (I): 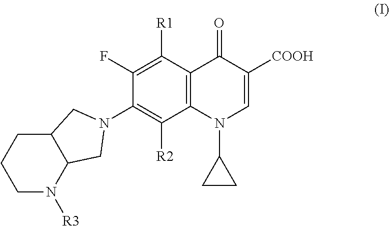
wherein:
R1 is H, amino, C1-C4 alkylamino, or C1-C4 dialkylamino
R2 is F, OMe, or H;
R3 is methyl, C2-C4 alkyl, or H; and
at least one of R1 and R3 is not H;
and a pharmaceutically acceptable vehicle.

2. A topical composition according to claim 1, wherein the compound of formula (I) is 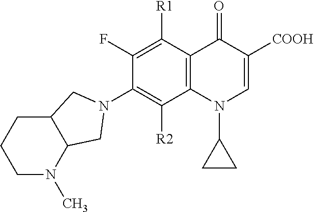
wherein:
R1 is H, amino, C1-C4 alkylamino, or C1-C4 dialkylamino; and
R2 is F, OCH₃, or H.

3. A topical composition according to claim 1, wherein the compound of formula (I) is 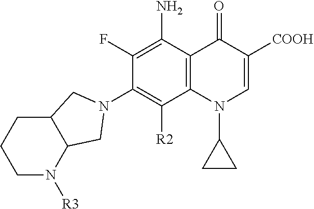
wherein:
R2 is F, OCH₃, or H; and
R3 is CH₃, C2-C4 alkyl, or H.

4. A topical composition according to claim 1, wherein the compound of formula (I) is selected from the group consisting of:

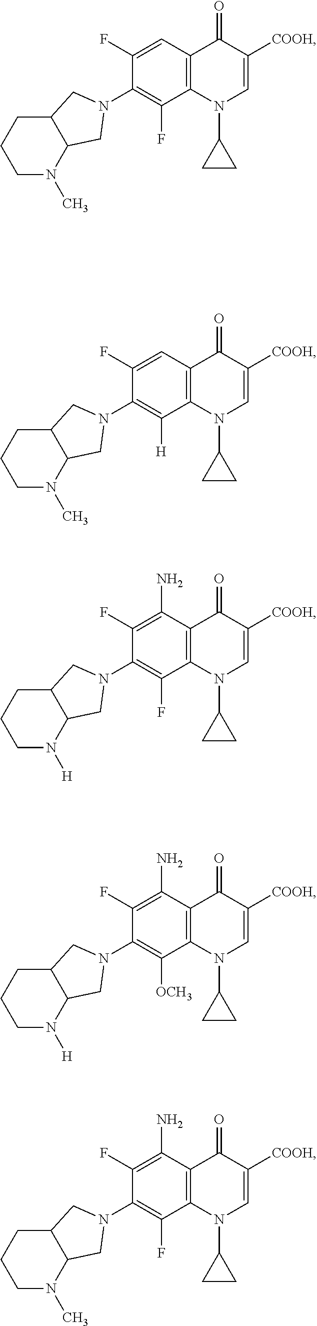

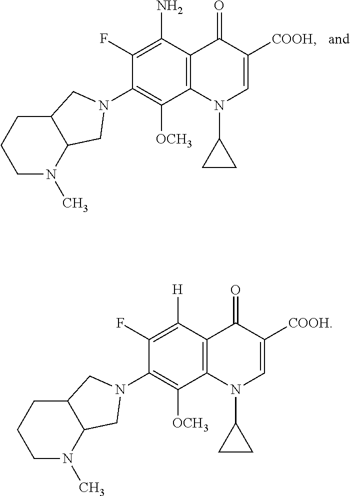

5. A topical composition according to claim 1, further comprising an anti-inflammatory agent.

6. A topical composition according to claim 5, wherein said anti-inflammatory agent is selected from the group consisting of:
steroidal and non-steroidal anti-inflammatories.

7. A topical composition according to claim 5, wherein said anti-inflammatory agent is selected from the group consisting of:
dexamethasone, prednisolone, rimexolone, nepafenac, and cilomilast.

8. A topical composition according to claim 1, wherein said compound is present at a concentration of from 0.05% to 0.3% w/v.

9. A topical composition according to claim 9, wherein said compound is present at a concentration of about 0.3% w/v.

10. A method of treating or preventing ophthalmic infections, which comprises topically applying a pharmaceutically effective amount of the composition of claim 1 to an ophthalmic tissue.

11. A method of treating or preventing ophthalmic infections, which comprises topically applying a pharmaceutically effective amount of the composition of claim 4 to the affected ophthalmic tissue.

12. A method according to claim 10, wherein said composition comprises said compound at a concentration of from 0.05% to 0.3% w/v.

13. A method according to claim 12, wherein said composition comprises said compound at a concentration of about 0.3% w/v.

14. A method according to claim 10, wherein said composition is applied from one to three times daily.

15. A method according to claim 10, wherein said composition is applied one time a day.

16. A method according to claim 10, wherein said composition comprises an anti-inflammatory agent.

17. A method according to claim 10, wherein said anti-inflammatory agent is selected from the group consisting of:
steroidal and non-steroidal anti-inflammatories.

18. A method according to claim 17, wherein said anti-inflammatory agent is selected from the group consisting of:
dexamethasone, prednisolone, rimexolone, nepafenac, and cilomilast.
